(19) United States
(12) Patent Application Publication
(10) Pub. No.: US 2018/0116178 A1
(43) Pub. Date: May 3, 2018

(54) ABSORBENT ARTICLE FOR PET ANIMALS

(71) Applicant: UNICHARM CORPORATION, Ehime (JP)

(72) Inventors: Daisuke KOMATSUBARA, Kagawa (JP); Hiroki YAMAMOTO, Kagawa (JP)

(21) Appl. No.: 15/568,311

(22) PCT Filed: Feb. 19, 2016
(86) PCT No.: PCT/JP2016/054813

§ 371 Date: Oct. 20, 2017

(30) Foreign Application Priority Data
May 25, 2015 (JP) 2015-105735

(51) Int. Cl. A01K 23/00; A61F 13/56

(52) U.S. Cl. CPC A01K 23/00; A61F 2013/15186; A61F 13/56

(57) ABSTRACT

An absorbent article for pet animals which makes possible to provide relatively easy handling for using the article. The absorbent article for a pet animal having a first side edge portion and a second side edge portion. The first side edge portion has a side seam coupling a first side edge portion of a ventral region and a first side edge portion of a dorsal region together. The second side edge portion is provided with a coupling means for coupling a second side edge portion of the ventral region to a second side edge portion of the dorsal region.

### TECHNICAL FIELD

The present disclosure relates to an absorbent article for pet animals such as dogs and cats that are used to absorb and contain body waste from the pet animals.

### BACKGROUND

Conventionally, absorbent articles are known including a diaper for applying on a pet animals including dogs. For example, Patent Literature 1 discloses a diaper of pull-on type for a dog having a ventral region, a dorsal region, an intermediate region extending between the ventral and dorsal regions, a liquid-permeable interior sheet layer, a liquid impermeable exterior sheet layer, and an absorbent core interposed between the interior and exterior sheet layers, wherein lateral edges of the ventral region and lateral edges of the dorsal regions are respectively connected to each other to define a waist-hole and a pair of leg holes.

Moreover, for example, Patent Literature 2 discloses a diaper of open-type for a dog having a liquid-permeable interior sheet layer, a liquid-impermeable exterior sheet layer, and an absorbent core interposed between the interior and exterior sheet layers, wherein the diaper is applied on a dog with the article secured around the dog's body.

### CITATION LIST

### Patent Literature

PTL 1: JP2012-205561 A

PTL 2: JP2013-9657 A

### SUMMARY OF INVENTION

### Technical Problem

The diaper for a dog disclosed in PTL 1 is mainly directed to use for a female dog. Since the diaper has a pull-on type, the diaper is applied on a dog in standing posture by slipping the dog's both rear legs through a pair of leg holes almost at the same time while slipping the dog' s hindquarters through the waist hole, and whereby the diaper makes it possible to cover the dog's hindquarters to absorb and contain body waste from the dog.

The diaper for a dog disclosed in PTL 2 is mainly directed to use for a male dog. The diaper is applied on the dog's hindquarters by engaging both end portions thereof to each other with the dog's hindquarters encircled with the diaper, and whereby the diaper makes it possible to prevent body waste from the dog from leaking out.

However, dogs dislike to have their legs lifted by a handler while moving the dog's legs in supine posture or in standing posture when applying the diaper on the dogs. This makes it difficult it for a handler to apply the diaper on the dogs.

In particular, the rear legs of dogs respectively have the rearward protruding or bending heels positioning higher than the insteps of the rear legs. This makes it difficult for a handler to slip the rear legs through a pair of leg holes of the diaper.

An object of the present invention is to provide an improved absorbent article for pet animals that is applied thereon by a relative easy handling procedure.

### Solution to Problem

To achieve the object, the present invention is featured in the following aspect:

An absorbent article has a longitudinal direction, a transverse direction orthogonal to the longitudinal direction, a body-facing surface, a non-body-facing surface opposed to the body-facing surface. The article includes a ventral region, a dorsal region, an intermediate region extending in the longitudinal direction between the ventral region and the dorsal region, and an absorbent core extending from the intermediate region to the ventral region.

The absorbent article further has a first side edge portion and a second side edge portion respectively extending in the longitudinal direction. The intermediate region has a tail hole through which a tail of the pet animal is slipped. The absorbent core is spaced apart from the tail hole toward the ventral region. The first side edge portion has both side edge portions of the ventral and dorsal regions coupled to each other along a side seam made on the first side edge portion. The second side edge portion is provided with a coupling means for detachably coupling both side edge portions of the ventral and dorsal regions to each other.

The absorbent article for pet animals may include the following embodiments.

(1) The coupling means includes a fastening tab.

(2) The fastening tab is provided on the ventral region.

(3) The ventral region is contractibly provided with first elastic elements extending in the transverse direction.

(4) The ventral region has a proximal edge straightly extending in the transverse direction, and the intermediate region has both side edges straightly extending in the longitudinal direction.

(5) The article further includes a ventral panel forming the ventral region, a dorsal panel forming the dorsal region, and an absorbent panel including the absorbent core and extending in the longitudinal direction between the ventral panel and the dorsal panel.

(6) A dimension of the ventral panel in the longitudinal direction is smaller than a dimension of the absorbent panel in the transverse direction.

(7) The ventral region has an inelastic region in which the absorbent core is present and the first elastic elements are not present.

(8) The first side edge portion is provided with a fastening means for fastening together part of the ventral region and part of the dorsal region with the ventral and dorsal regions coupled to each other

(9) The absorbent panel is contractibly provided with a second elastic element extending in the longitudinal direction along each of both side edges of the absorbent core.

### Advantageous Effects of Invention

With the absorbent article for pet animals according to the embodiments of the present invention, both side edge portions forming the first side edge portion is pre-coupled along the side seam to thereby define a leg hole through which one rear leg of the pet animal is slipped, while both side edge portions forming the second side edge portion are possible to be coupled each other by the coupling means so that the leg hole to encircle to the other rear leg of the pet animal may be defined when the absorbent article is applied on the pet animal. This makes it possible to apply the absorbent article on the pet animal smoothly even when the pet animal is in standing posture.

The embodiment (1) described above makes it possible to easily do the handling procedure for coupling the ventral region to the dorsal region when applying the absorbent article on the pet animal because the coupling means includes the fastening tab.

The embodiment (2) described above makes it possible to stably engage the ventral region with the dorsal region with the fastening tab in the process of handling for applying compared with when the fastening means is provided on the dorsal region because the coupling means is provided on the ventral region.

The embodiment (3) described above makes it possible to fit the ventral region to the pet animal's body because the first elastic elements extending in the transverse direction are provided on the ventral region.

The embodiment (4) described above makes it possible to inhibit the groins of the pet animal from coming in contact with the absorbent article when the pet animal walks because the proximal edge of the ventral region straightly extending in the transverse direction and both side edges of the intermediate region straightly extending in the longitudinal direction and their edges cross each other at the upper edges of the leg holes.

The embodiment (5) described above makes it possible to fit the peripheral edges of both leg holes to the groins of the pet animal, respectively, because the absorbent article includes the absorbent panel between the ventral and dorsal regions and the second elastic elements lying in the absorbent panel and being stretchable/contractable in the longitudinal direction without being influenced by a contractible force of the first elastic elements.

The embodiment (6) described above does not make it difficult for the pet animal to move the rear legs due to the groins of the rear legs coming in contact with the ventral region, because a dimension in the transverse direction of the ventral region is smaller than a dimension in the transverse direction of the absorbent panel.

The embodiment (7) described above makes it possible to inhibit the occurrence of gathers such as lowering the absorption performance of the absorbent core because the ventral region has the inelastic region in which a contractible force of the first elastic elements does not exert and the absorbent core is present.

The embodiment (8) described above makes it possible to pull together both side edge portions of the ventral and dorsal region forming the first side edge portion, similarly to the second side edge portion, because the first side edge portion is provided with the fastening means.

The embodiment (9) described above makes it possible to prevent the leakage of body waste from both side edge portions of the absorbent panel to the pet animal's body because the second elastic elements are provided along both side edges of the absorbent core.

### BRIEF DESCRIPTION OF DRAWINGS

The drawings illustrate specific embodiments of the present invention including optional and preferred embodiments as well as essential features of the invention. It should be noted here that there are shown a dog as an example of pet animals and a diaper as an example of absorbent articles.

FIG. 1 is a perspective view of a diaper according to a first embodiment of the present invention.

FIG. 2 is a partially cutaway and opened plan view of the diaper in which respective elastic elements are provided are stretched at the maximum in the longitudinal and transverse directions, i.e. ; to the extent that gathers formed in the diaper under a contractible force of the respective elastic elements disappear.

FIG. 3 is a cross-sectional view along line III-III in FIG. 2.

FIG. 4 is a perspective view from one side of the dog wearing the diaper.

FIG. 5 is a perspective view from the other side of the dog wearing the diaper.

FIG. 6 (a) to (c) are respectively perspective views showing a handling procedure for applying the diaper on the dog.

FIG. 7 is a view similar to FIG. 2 showing a diaper according to a second embodiment of the present invention.

FIG. 8 is a perspective view from one side of the dog wearing the diaper according to the second embodiment of the present invention.

### DESCRIPTION OF EMBODIMENTS

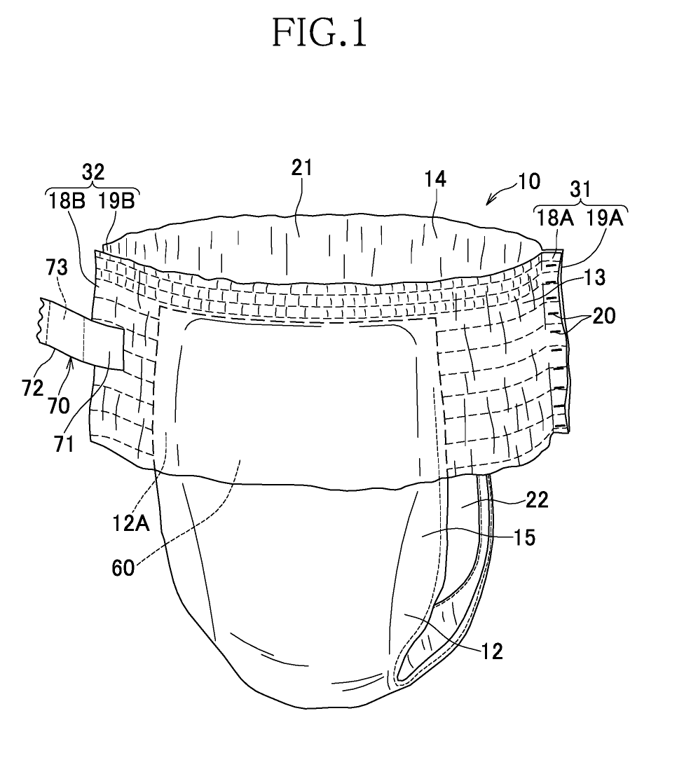

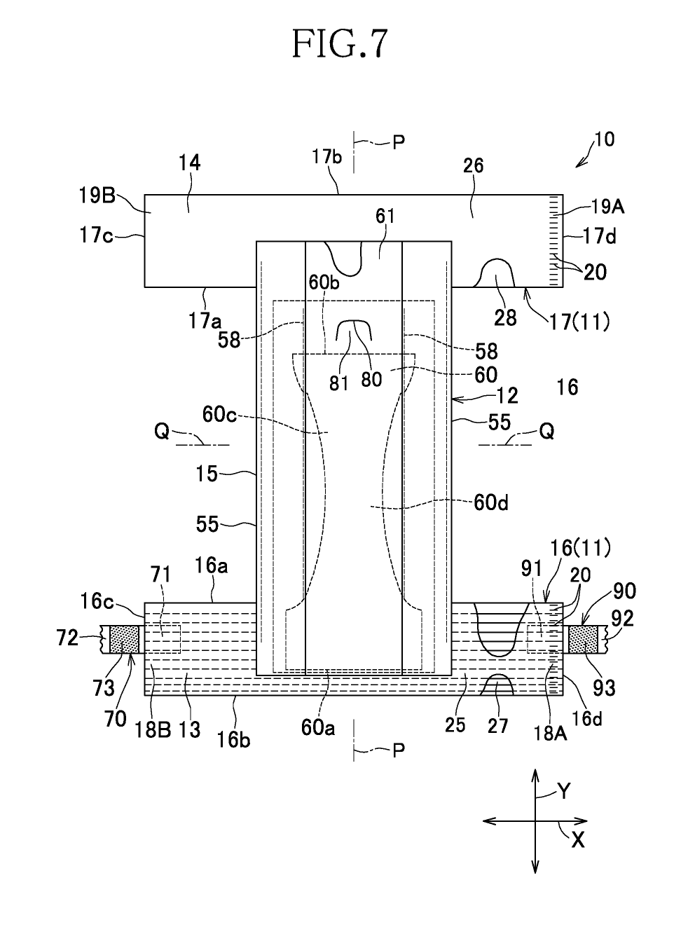

The embodiments described below relate to a diaper as an example of absorbent articles as illustrated in FIGS. 1 through 8, including both optional and preferred features as well as those features which are essential of the present invention.

### First Embodiment

With reference to FIGS. 1 through 4, a diaper 10 for a dog 5 has a longitudinal direction Y, a transverse direction X being orthogonal to each other, a first axis P bisecting a dimension in the transverse direction X, a second axis Q bisecting a dimension in the longitudinal direction Y, a body-facing surface, and a non-body-facing surface opposed to the body-facing surface.

The diaper 10 includes a annular waist panel 11, an absorbent panel 12 attached to the body-facing surface of the waist panel 11, a ventral region 13, a dorsal region 14 and an intermediate region 15 extending in the longitudinal direction Y between the ventral and dorsal regions 13 and 14. The waist panel 11 includes a ventral panel 16 forming the ventral region 13 and a dorsal panel 17 forming the dorsal region 14.

The ventral and dorsal panels 16 and 17 respectively cross the absorbent panel 12 and have a transversely long rectangle defined by proximal edges 16a and 17a extending in the transverse direction X, distal edges 16b and 17b opposed to the proximal edges 16a and 17a in the longitudinal direction Y and extending in the transverse direction X, and both side edges 16c and 16d, 17c and 17d extending in the longitudinal direction Y between proximal edges 16a and 16b, 17a and 17b. Aside edge portion 18A of the ventral region 13 and a side edge 19A of the dorsal region 14 are pre-coupled to each other along a side seam 20 continual in the longitudinal direction Y. The side seam 20 is made by well-kwown bonding means, for example, thermal welding techniques including heat embossing/debossing and ultrasonic processing.

On the other hand, a side edge portion 18B of the ventral region 13 and a side edge portion 19B of the dorsal region 14 are not pre-coupled to each other, but the side edge portion 18B of the ventral region 13 is provided with a coupling means including a fastening tab 70. The absorbent article 10 has a first side edge portion 31 that the side edge portion 18A of the ventral region 13 and the side edge 19A of the dorsal region 13 are pre-coupled to each other along the side seam 20, and a second side edge portion 32 that the side edge portion 18B of the ventral region 13 and the side edge portion 19B of the dorsal region 14 is possible to be detachably coupled to each other with the fastening tab 70. With reference to FIG. 1, one leg hole 22 is defined by coupling both side edge portions 18A and 19A on the side of the first side edge portion 31 and the other leg hole 22 (not shown) in the process of being defined by coupling both side edge portions 19A and 19B on the side of the second side edge portion 31.

The ventral panel 16 and the dorsal panel 17 respectively have flexible interior sheet layers 25 and 26 on the side of the body-facing surface and flexible exterior sheet layers 27 and 28 on the side of the non-body-facing surface. The interior and exterior sheet layers 25 and 26, 27 and 28 include a liquid-impermeable SMS (spun-bonded/melt-bonded/spun-bonded) fibrous nonwoven fabric, a spun-bonded fibrous nonwoven fabric, or a laminated sheet layer of them, each having a mass per unit area of 15 g/m² to 30 g/m². The sheet layers 25 and 26, 27 and 28 are respectively joined to each other by hot melt adhesive continually applied to an interior surface of one of the sheet layers 25 and 26 and one of the sheet layers 27 and 28 or by thermal welding techniques.

The ventral panel 16 is provided with a plurality of strand-or string-like first elastic elements 41 extending in the transverse direction X in a contractible manner under tension between the interior and exterior sheet layers 25 and 27, thereby being elasticized at least in the transverse direction X. It should be noted here that the interior and exterior sheets 25, 27 may be joined only by hot melt adhesive applied to the entire peripheries of the first elastic elements.

The first elastic elements 41 includes a plurality of distal elastic elements 41A extending in the transverse direction X along the distal edge 17a of the ventral panel 16, and a plurality of proximal elastic elements 41B extending in the transverse direction X adjacently to the distal elastic elements 41A and along the proximal edge 16a of the ventral panel 16. The proximal elastic elements 41B is not overlapped with an absorbent core 60 (described later) contained in the absorbent panel 12 in a planar view (i.e., in the thickness direction) wherein the proximal elastic elements 41B extend from respective side edges 16c and 16d of the ventral panel 16 to the vicinities of respective side edges 60c and 60d of the absorbent core 60. In this way, the ventral panel 16 has an elastic region in which a contractible force of the proximal elastic elements 41B directly exerts and an inelastic region in which the contractile force of the proximal elastic elements 41B does not exert and the absorbent core 60 is present. Since the contractible force of the proximal elastic elements 41B does not exert the absorbent core 60 in the inelastic region, folds or gathers such as lowering the absorption performance of the absorbent core 60 should not occur thereon.

The absorbent panel 12 is formed in a longitudinally long rectangle having a front end portion 12A fixed on the body-facing surface of the ventral panel 16, a rear end portion 12B fixed on the body-facing surface of the dorsal region 17 and an intermediate portion 12C extending in the longitudinal direction Y between the front and rear end portions 12A and 12B. The absorbent panel 12 includes a flexible cover sheet layer 50, an absorbent core 60 put on the body-facing surface of the cover sheet layer 50, a liquid-permeable flexible bodyside liner 61 covering at least an absorbing surface of the absorbent core 60, and a flexible liquid-barrier sheet layer 62. In the present embodiment, although the absorbent article 10 is constructed of three separate panels 12, 16 and 17, it may be constructed of a singly and integrally formed annular panel having regions corresponding to the ventral and dorsal regions 13 and 14, or a singly and integrally formed panel having regions corresponding to the ventral, dorsal and intermediate regions 13, 14 and 15.

The absorbent core 60 is contoured by both end edges 60a and 60b and both concave side edges 60a and 60b, and constructed of a mixture of superabsorbent polymer (SAP) particles having a water-absorption capacity at least 10 times as high as its own mass, wood pulp fluff and optionally a small amount of thermoplastic fibers. The absorbent core 60 may be wrapped with a wrapping sheet layer (not shown) made of a hydrophilic, liquid-diffusive fibrous nonwoven fabric or tissue paper layer, which may be joined to at least one of the bodyside liner 6 1 and the liquid-barrier sheet layer 62 with hot melt adhesive.

The cover sheet layer 50 has an intermediate portion 51 on which the absorbent core 60 is put and both side portions 52. Both side portions 52 are respectively folded inward in the transverse direction X and has both end portions secured to the intermediate portion 51, proximal portions 55 secured to the intermediate portion 51 and the liquid-barrier sheet layer 62 while extending in the longitudinal direction Y between both the end portions, and distal portions 56 lying inside the proximal portions 55. The distal portions 56 each may be lain outside the proximal portions 55 without folding the cover sheet layer inward, as necessary. The distal portions 56 each have distal edge sleeves 56a in which elastic elements 58 each are provided in a contractible manner under tension. The distal portions 58 can be spaced away from the flexible bodyside liner 61 toward the body of a dog, and whereby formed are leakage-barrier cuffs to prevent the dog' s body waste from leaking out.

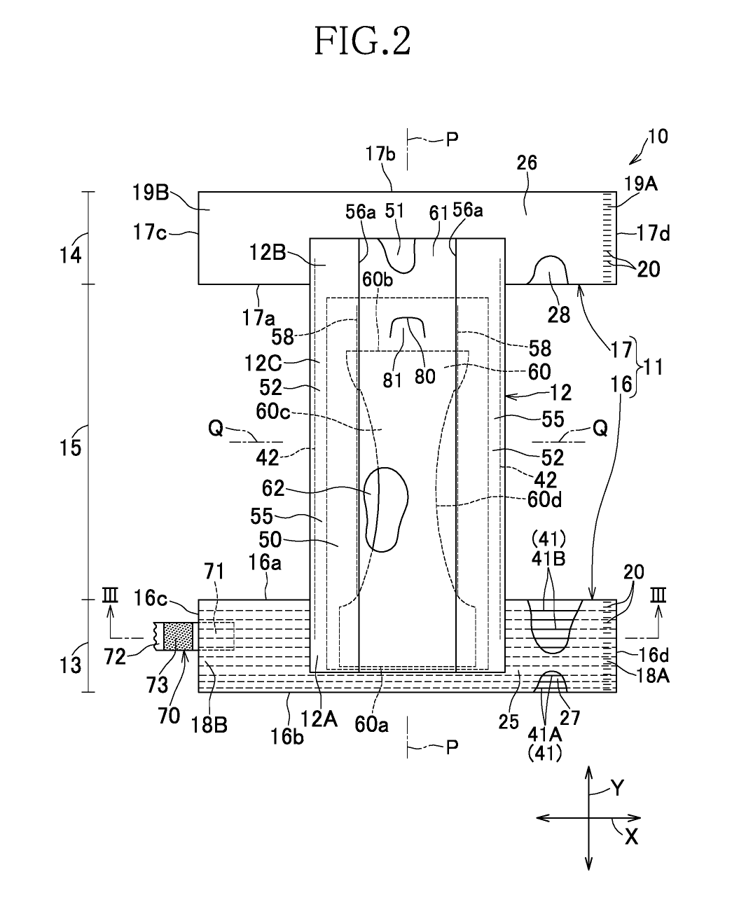

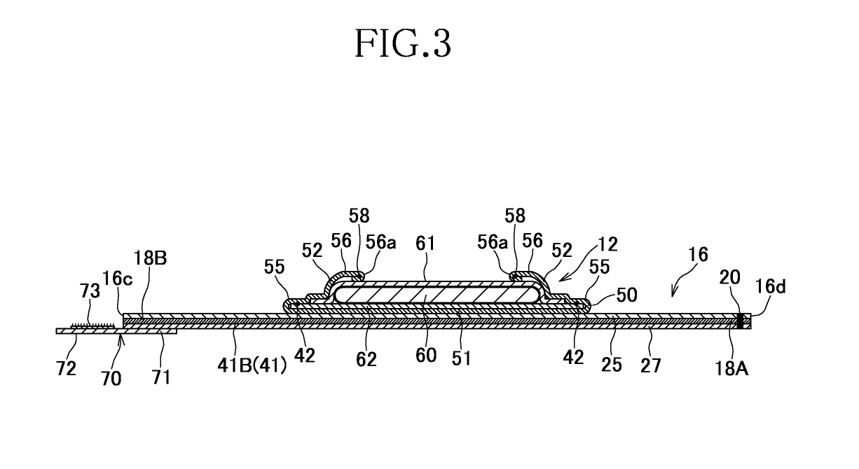

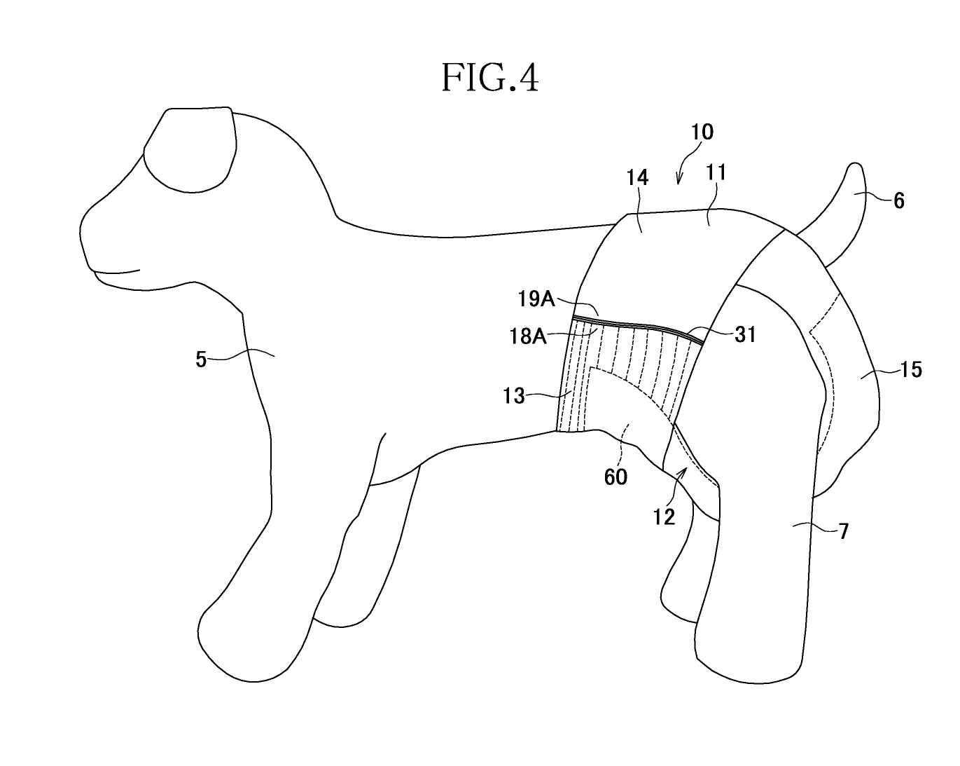

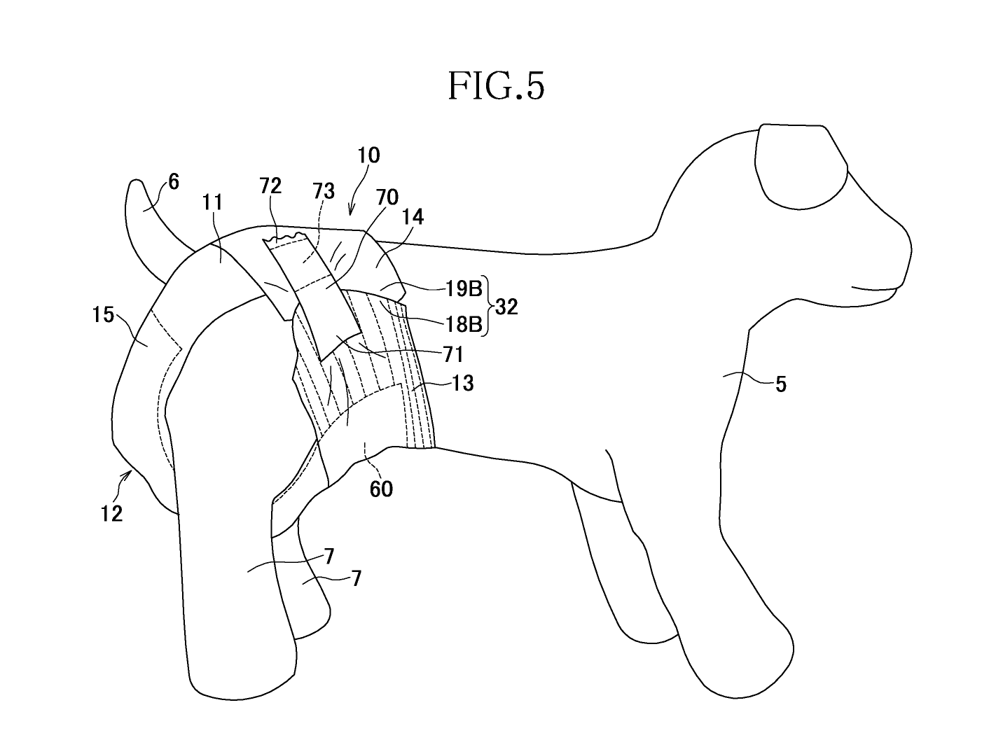

With reference to FIG. 2 and FIG. 3, second elastic elements 42 extending in the longitudinal direction Y each are attached in a contractible manner under tension along the proximal portions 55 of the cover sheet layer 50 that are parts of both side portions 52 located outside in the transverse direction X of both side edges 60c and 60d of the absorbent core 60, and whereby both side portions 52 are formed with both side elastic regions contractible in the longitudinal direction Y that a contractible force of the second elastic elements 42 exert. The side elastic regions fit to the thighs of the rear legs 7 of the dog 5 (see FIGS. 4, 5) when the absorbent article 10 is applied on the body of the dog 5. This makes it possible to prevent the positional displacement of both side portions 52 with respect to the dog's body due to the movements of the rear legs 7 and the leakage of the dog's body waste due thereto from occurring.

With reference to FIG. 1 and FIG. 2, the absorbent article 10 is provided with a coupling means including a fastening tab 70 attached to the interior surface of a side edge potion 18B of the ventral region 13 forming the second side edge portion 32 of the absorbent article 10. The fastening tab 70 has a proximal portion 71 connected to the second side edge portion 32 of the absorbent article 10 and a distal portion 72 extending outward in the transverse direction X from the side edge of the ventral region 13(i.e., the side edge 16c of the ventral panel 16), and the distal portion 71 is provided with an engaging portion 73 on its interior surface. The engaging portion 73 is constructed of, for example, a fastening element including multiple hooks of well-known mechanical fasteners that is relesably engageable with an exterior surface of a side edge portion 19B of the dorsal region 14 and a fastening element supporting hook sheet secured on the distal portion 72.

The engaging portion 73 is releasably engageable with the exterior sheet layer 28 of a fibrous nonwoven fabric defining an exterior surface of the dorsal region 14. A landing sheet layer having multiple loops of well-known mechanical fasteners may be disposed on a predetermined zone of the exterior surface of the dorsal region 14 to stably engage the distal portion 72 with the predetermined zone. When the exterior sheet layer 28 is composed of a plurality of layers and the outermost layer thereof is formed of a plastic film, an adhesive zone may be applied on the interior surface of the distal portion 72 of the fastening tab 70 instead of the hooks so as to detachably adhere the adhesive zone on the plastic film. The proximal portion 71 of the fastening tab 70 may be secured between portions of the interior and exterior sheets layers 25 and 27 forming the side edge portion 18B of the ventral region 13, or on the exterior surface of the side edge portion 18B instead of the interior surface thereof. A grasping portion at the outer side of the fastening tab 70 has preferably a suitable size that is easy to be grasped by a handler such that the fingers of the handler do not come in contact the engaging portion 73 when the grasping portion is grasped by the handler. The fastening tab 70 may be two or more and have a size such that extends along the almost entire length in the longitudinal direction Y of the side edge portion 18B. Moreover, the fastening tab 70 may be elastically stretchable and contractible, or in a state folded in a plurality of layers so as to be extendable, or in a state that the distal portion 72 is dividable in a plurality of pieces on which the engaging portion 73 are respectively provided.

With reference to FIG. 2, the diaper 10 is provided in an area facing the tail 6 of a dog 5 in the intermediate region 15 with a generally U-shaped cutting line 80 formed of perforations for formation of a tail hole through which the tail 6 may be slipped. The cutting line 80 is passed through the bodyside liner 61, the liquid-barrier sheet 62 and the cover sheet 50 overlapped with each other in a planar view. The cutting line 80 is located between the distal portions 56a forming the leakage-barrier cuffs, the leakage-barrier cuffs are spaced away from the bodyside liner 61 and put in contact with the tail 6, and the tail 6 is slipped through a tail hole pre-formed along the cutting line 80 when the absorbent article 10 is applied on the dog 5. The tail hole, through which the tail 6 is stuck, makes it possible to prevent the tail 6 from getting out of the tail hole due to the movements of the tail 6. The absorbent core 60 is spaced apart from the tail hole toward the ventral region 13. More specifically, a rear end edge 60b of the absorbent core 60 is spaced apart from the cutting line 80 to form the tail hole toward the absorbent core 60, and a non-present area of the absorbent core 60 is around the tail hole. When the absorbent article 10 is applied on the dog 5, as described later, an area of the absorbent article 10 around the tail hole is pulled toward the tail 6 with the tail 6 slipped through the leg hole on the side of the first side portion 31 of the absorbent article 10. For this reason, although the area of the absorbent article 10 around the tail hole needs to be deformed so that the tail 6 may be easily slipped through the tail hole, the present of the absorbent core 60 around the tail hole may relatively stiffen the area of the absorbent article 10 around the tail hole and make it difficult the deformation of the area of the absorbent article 10 around the tail hole. Thus, the non-present area of the absorbent core 60 in which the area of the absorbent article 10 around the tail hole is preferably constructed of flexible sheet elements alone. Such construction facilitates the deformation of the area of the absorbent article 10 around the tail hole and the handling of applying the absorbent article 10 on the dog 5.

With reference to FIG. 4 and FIG. 5, in a state in which the absorbent article 10 is applied on the dog 5, for the first side edge portion 31, the side edge portion 18A of the ventral region 13 and the side edge portion 19A of the dorsal region 14 are pre-coupled to each other along the side seam 20, and for the second side edge portion 32, the side edge portion 18A of the ventral region 13 and the side edge portion 19A of the dorsal region 14 are coupled to each other by engaging the engaging portion 73 of the fastening tab 70 attached to the side edge portion 18B of the ventral region 13 with the exterior surface of the side edge portion 19B of the dorsal region 14.

For conventional diapers for mainly female dogs of a type in which both side edge portions of the ventral region and both side edge portions of the dorsal region are respectively coupled to each other by a coupling means including a fastening tab attached to one of the ventral and dorsal regions, when the diaper is applied on the dog in standing posture, the respective both side edge portions needs to be coupled to each other almost at the same time to properly apply the diaper on the dog. If the dog is forcibly put in supine posture to stably apply the diaper on the dog, the dog may make the movements such as thrashing about or striking out the rear legs while disliking such supine posture. This makes it difficult to apply the diaper on the dog in a smooth manner.

For conventional diapers for mainly female dogs of a type in which both side edge portions of the ventral region and both side edge portions of the dorsal region are respectively coupled to each other by the side seams made along the side edge portions, when the diaper is applied on the dog in standing posture, both rear legs of the dog having the heels positioning higher than the insteps make it difficult to smoothly slip the rear legs through the leg holes of the diaper. If the diaper is forcibly lifted to slip the legs through the leg holes, the leg holes may be torn because of being caught by the heels. When the diaper is applied on the dog in supine posture, the dog dislikes not only assuming such a posture, but also having the rear legs caught by a handler. This makes it difficult to smoothly apply the diaper on the dog. Unlike the human body, the dog's rear legs are orthogonal to the dog' s body in any of a standing posture and a supine posture, so that the handler needs to apply the diaper on the dog while lifting the rear legs in a direction orthogonal to the legs. In such handling to apply the diaper on the dog, portions of the diaper to be caught by the handler needs to be changed, which is quite troublesome to the handler.

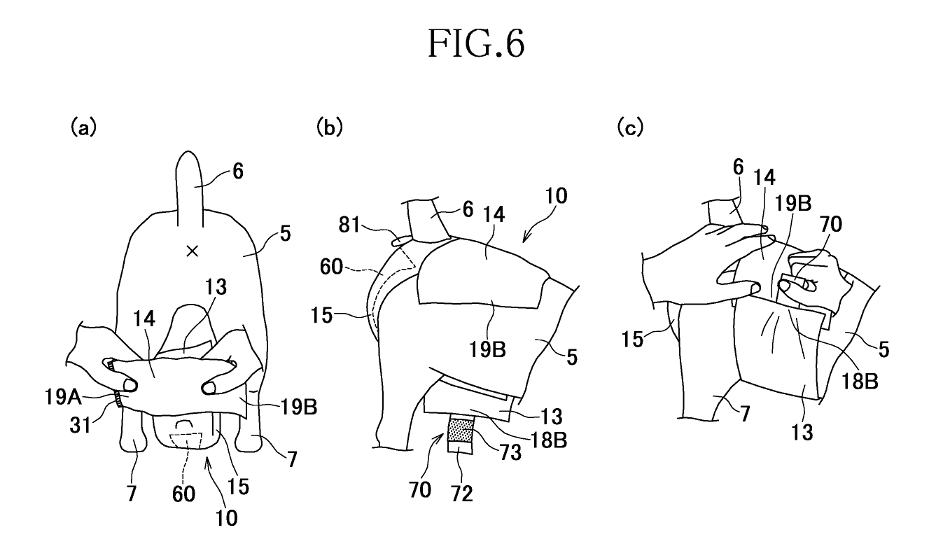

FIGS. 6 (a)-(c) shows a handling procedure for applying the diaper on the dog. With reference to FIG. 6 (a), for example, first, one of the rear legs 7 of the dog 5 is slipped through the leg hole 22 defined on the side of the first side edge portion 31 that is closed by the side seam 20 b. At this time, the second side edge portion 32 is in an open state, i.e., in a state that the side edge portion 18B of the ventral region 13 and the side edge portion 19B of the dorsal region 14 are not coupled to each other. Thus, the side edge portion 18B of the ventral region 13 and the side edge portion 19B of the dorsal region 14 can be opened while being widely spaced apart from each other. Referring to FIG. 6 (b), next, the intermediate region 15 is put on the rump and the tail 6 is slipped through a tail hole pre-formed in the intermediate region 15.

With reference to FIG. 6 (c), following the handling procedure described above, the dorsal region 14 is put on the back of the dog 5, the ventral region 13 is pulled up toward the back of the dog 5 so that the side edge portions 18B may be overlapped on the side edge portion 19B and the distal portion 72 of the fastening tab 70 is pressed against the exterior surface of the side edge portion 19 to engage the engaging portion 73 with the side edge portion 19. Thus, the side edge portions 18B and 19B are coupled to each other to encircle the other of the rear legs of the dog 5, and as a result, a leg hole 22 (not shown) is defined on the second side edge portion 32 side.

In such a series of the handling procedures, since the second side edge portion 32 is in the open state, one of the rear legs 7 is easily slipped through the leg hole on the first side edge portion 31 side, and also when the coupling of the second side edge portion 32 is handled, the diaper 10 should not be off from the body of the dog 5 by sliding down or dropping even if the dog 5 makes the movements such as the dog 5 dislikes to have the rear legs 7 caught by a handler. This allows the handler to handle so that the diaper 10 may be applied on the dog 5 smoothly.

With reference again to FIG. 2, the diaper 10 is constructed of a rectangular waist panel 11 and a rectangular absorbent panel 12, the proximal edge 16a of the ventral region 13 straightly extend in the transverse direction X and both side edges 16c and 16d of the intermediate region 15 straightly extends in the longitudinal direction Y so as to be orthogonal to each other. If the proximal edge 16a of the ventral region 13 and/or both side edges of the absorbent panel 12 are curved, the walk of the dog 5 may be hindered because the groins of the rear legs 7 moving in the front-rear direction come in contact with the curved proximal edge 16a of the ventral region 13 and/or the side edges of the absorbent panel 12. However, according to the present embodiment in which the proximal edge 16a of the ventral region 13 and/or both side edges of the absorbent panel 12 are orthogonal to each other, since the waist panel 11 and the absorbent panel 12 are not positioned adjacently to the groins in a state that the diaper 10 is put on the dog 5, the walk of the dog 5 should not be hindered because of the contact of the diaper 10 against the rear legs 7. Moreover, a dimension in the longitudinal direction Y of the ventral panel 16 (i.e., a dimension in the longitudinal direction Y of the ventral region 16c) is smaller than a dimension in the transverse direction X of the absorbent panel 12. In conventional diapers, the dimension of the former is larger than the dimension of the latter so that the ventral region of the dog 5 may entirely be covered by the ventral region 13. However, in the present embodiment, since the dimension of the former is smaller than the dimension of the latter, the ventral region 13 should not come in contact with the groins, and thus the walk of the dog 5 should not be hindered.

The fastening tab 70 is attached to the ventral region 13 in the present embodiment. With reference to FIG. 6(b), if the fastening tab 70 is attached to the dorsal region 14, the fastening tab 70 is brought into a state such as hanging down from the back of the dog 5 by its own weight, when the side edge 18B of the ventral region 13 and the side edge portion 19B of the dorsal region 14 on the second side edge portion 32 side are coupled to each other in the handling process for applying the diaper 10 on the dog 5. This may make it difficult for a handler to grasp the fastening tab 70. Also, the engaging portion 73 needs to be pressed against the side edge portion 19B of the dorsal region 14 on the side of the dog 5, here it is difficult to do so and to stably engage the engaging portion 73 to the side edge portion 19B of the dorsal region 14. As in the present embodiment, the fastening tab 70 may be easily grasped because the fastening tab 70 is attached to the ventral region 13, and also the engaging portion 73 can be pressed against the side edge portion 19B of the dorsal region 14 to stably engage therewith.

### Second Embodiment

The basic structure of the diaper 10 according to a second embodiment of the present invention is substantially the same as that of the first embodiment of the present invention. Accordingly, for the second embodiment, only the structure of a diaper different from the diaper 10 according to the first embodiment is hereinafter described.

A diaper 10 according to the second embodiment, in addition to the fastening tab 70 attached to the side edge portion 18B of the ventral region 13, a fastening tab 90 is attached to the side edge portion 18A of the ventral region 13. The fastening tab 90 has a proximal portion 91 on the side edge portion 18A, a distal portion 92 extending outward in the transverse direction X from the side edge 16d of the ventral region 13, and an engaging portion 93 provided on the interior surface of the distal portion 92. The fastening tab 90 has a grasping portion adjacent to the engaging portion 93.

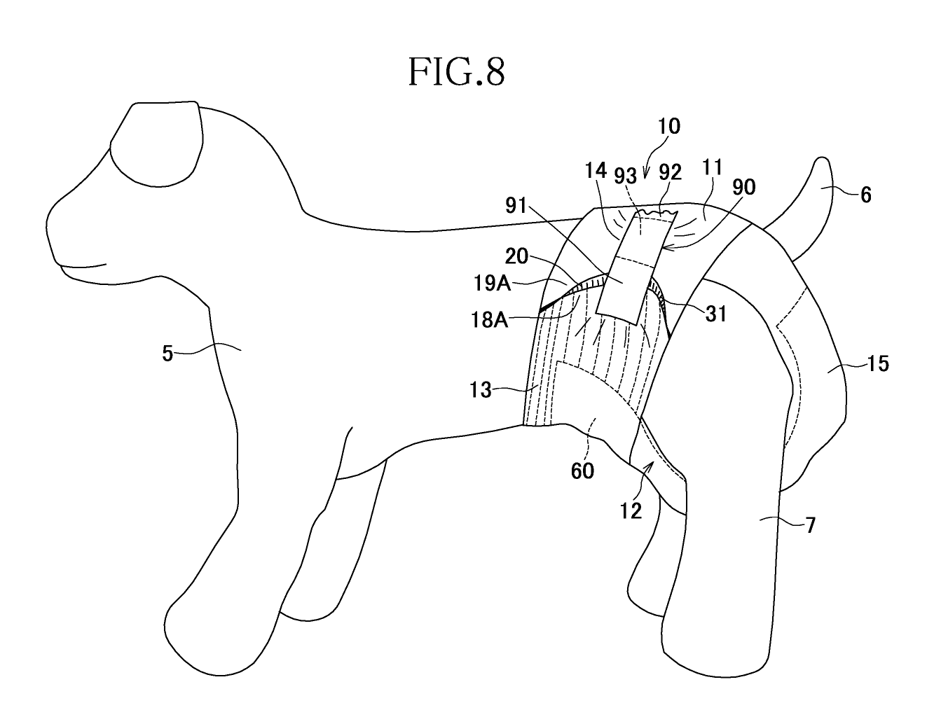

With reference to FIG. 4, FIG. 5 and FIG. 8, on the first side edge portion 31, the side edge portion 18A of the ventral region 13 and the side edge portion 19A of the dorsal region 14 is coupled to each other by the side seam 20, while on the second side edge portion 32, by pulling of the fastening tab 70, the side edge portion 18B of the ventral region 13 and the side edge portion 19B of the dorsal region 14 are coupled to each other so that the side edge portion 18B of the ventral region 13 may be pulled to the side edge portion 19B of the dorsal region 14, in other words, the side edge portions 18B and 19B are pulled together toward the body of the dog 5, and thus they may be overlapped to each other. Whereas, on the first side edge portion 31, since the side edge portions 18A, 19A are pre-coupled to each other along the side seam 20, they are not pulled together toward the body of the dog 5. As a result, the first side edge portion 31 may be lowered compared with the second side edge portion 32 with respect to fitting to the body of the dog 5. Whereby the fitting maybe different between the first and second side edge portions 31 and 32, and thus even if the diaper 10 is properly applied on the dog 5, the positional displacement of the diaper 10 with respect to the body of the dog 5 may occur, or may cause the leakage of body waste from the dog 5 due to the loose fitting of the side edge portions 18A and 19A to the body of the dog 5. According the present embodiment, similarly to the second side edge portion 32, the side edge portion 31, i.e., the side edge portions 18A and 19A of the first side edge portion 31, may fit the body of the dog 5 by engaging the engaging portion 93 with the side edge portion 19B of the dorsal region 14 while pulling the distal portion 92 to the side of the dorsal region 14. This makes it possible to inhibit the loose fitting of the side edge portions 18A and 19A to the body of the dog 5.

Moreover, the attachment of the fastening tab 90 to the first side edge portion 31 makes it possible to adjust a size of the entirety of the diaper 10, for example, the diaper 10 makes it possible to apply the diaper 10 on the dog 5 in a state fitting the diaper 10 even to the relatively slim body of the dog 5. The fastening tab 90 may be attached to the side edge portion 19A of the dorsal region 14 instead of the side edge portion 18A of the ventral region 13. Here a plurality of the fastening tabs 90 as well as a single of the fastening tab 90 may be provided.

The structure elements of the diaper 10 are not limited to those described in the specification but other various types of materials widely used in the relevant technical field may be used without limitation unless otherwise stated. As used herein, the term such as “first” and “second” of the present specification and claims are used merely distinguish the similar elements, similar positions or other matters.

### REFERENCE SIGNS LIST

5 pet animal (dog)

6 tail

10 absorbent article (diaper)

12 absorbent panel

13 ventral region

14 dorsal region

15 intermediate region

16 ventral panel

16a proximal edge of ventral region

16b distal edge of ventral region

17 dorsal panel

18A one side edge portion of ventral region

18B other side edge potion of ventral region

19A one side edge portion of dorsal region

19B other side edge portion of dorsal region

20 side seam

31 first side edge portion

32 second side edge portion

41 first elastic elements

42 second elastic elements

60 absorbent core

70 fastening tab

90 fastening tab

X transverse direction

Y longitudinal direction

1. An absorbent article for a pet animal having a longitudinal direction and a transverse direction orthogonal thereto, and comprising a body-facing surface to face a body of a pet animal, a non-body-facing surface opposed thereto, a ventral region, a dorsal region, an intermediate region extending in the longitudinal direction between the ventral region and the dorsal region, and an absorbent core extending from the intermediate region to the ventral region,
wherein:
the absorbent article further has a first side edge portion and a second side edge portion respectively extending in the longitudinal direction,
the intermediate region has a tail hole through which a tail of the pet animal can be slipped,
the absorbent core is spaced apart from the tail hole toward the ventral region,
the first side edge portion has both side edge portions of the ventral and dorsal regions coupled to each other along a side seam made on the first side edge portion, and
the second side edge portion is provided with a coupling means for detachably coupling both side edge portions of the ventral and dorsal regions to each other.

2. The absorbent article for a pet animal according to claim 1, wherein the coupling means include a fastening tab.

3. The absorbent article for a pet animal according to claim 1, wherein the coupling means are provided on the ventral region.

4. The absorbent article for a pet animal according to claim 1, wherein the ventral region is contractibly provided with first elastic elements extending in the transverse direction.

5. The absorbent article for a pet animal according to claim 1, wherein the ventral region has a proximal edge straightly extending in the transvers direction and the intermediate region has both side edges straightly extending in the longitudinal direction.

6. The absorbent article for a pet animal according to claim 1 further comprising a ventral panel forming the ventral region, a dorsal panel forming the dorsal region, and an absorbent panel having an absorbent core, extending in the longitudinal direction and connecting the ventral panel and the dorsal panel.

7. The absorbent article for a pet animal according to claim 6, wherein a dimension of the ventral panel in the longitudinal direction is smaller than a dimension of the absorbent panel in the transverse direction.

8. The absorbent article for a pet animal according to claim 1, wherein the ventral region has an inelastic region in which the absorbent core is present and the first elastic elements are not present.

9. The absorbent article for a pet animal according to claim 1, wherein the first side edge portion is provided with a fastening means.

10. The absorbent article for a pet animal according to claim 1, wherein the absorbent panel is contractibly provided with a second elastic element extending in the longitudinal direction along each of both side edges of the absorbent core.

11. The absorbent article for a pet animal according to claim 2, wherein the coupling means are provided on the ventral region.

12. The absorbent article for a pet animal according to claim 2, wherein the ventral region is contractibly provided with first elastic elements extending in the transverse direction.

13. The absorbent article for a pet animal according to claim 3, wherein the ventral region is contractibly provided with first elastic elements extending in the transverse direction.

14. The absorbent article for a pet animal according to claim 11, wherein the ventral region is contractibly provided with first elastic elements extending in the transverse direction.

15. The absorbent article for a pet animal according to claim 2, wherein the ventral region has a proximal edge straightly extending in the transvers direction and the intermediate region has both side edges straightly extending in the longitudinal direction.

16. The absorbent article for a pet animal according to claim 3, wherein the ventral region has a proximal edge straightly extending in the transvers direction and the intermediate region has both side edges straightly extending in the longitudinal direction.

17. The absorbent article for a pet animal according to claim 11, wherein the ventral region has a proximal edge straightly extending in the transvers direction and the intermediate region has both side edges straightly extending in the longitudinal direction.

18. The absorbent article for a pet animal according to claim 4, wherein the ventral region has a proximal edge straightly extending in the transvers direction and the intermediate region has both side edges straightly extending in the longitudinal direction.

19. The absorbent article for a pet animal according to claim 12, wherein the ventral region has a proximal edge straightly extending in the transvers direction and the intermediate region has both side edges straightly extending in the longitudinal direction.

20. The absorbent article for a pet animal according to claim 13, wherein the ventral region has a proximal edge straightly extending in the transvers direction and the intermediate region has both side edges straightly extending in the longitudinal direction.
